(19) United States
(12) Patent Application Publication
(10) Pub. No.: US 2014/0017207 A1
(43) Pub. Date: Jan. 16, 2014

(54) ISOLATION, EXPRESSION AND GUIDED DIFFERENTIATION OF SELF-RENEWING PROGENITOR CELLS FROM ADULT HUMAN PANCREAS

(71) Applicant: The Johns Hopkins University, Baltimore, MD (US)

(72) Inventors: Steven D. Leach, Baltimore, MD (US); Meritxell Rovira-Clusellas, Baltimore, MD (US)

(73) Assignee: The Johns Hopkins University, Baltimore, MD (US)

(21) Appl. No.: 13/940,649

(22) Filed: Jul. 12, 2013

Related U.S. Application Data
(60) Provisional application No. 61/670,633, filed on Jul. 12, 2012.

(51) Int. Cl. C12N 5/071

(52) U.S. Cl. CPC C12N 5/0676
USPC 424/93.7; 435/34; 435/366

(57) ABSTRACT

The present invention relates to the field of pancreatic progenitor cells. More specifically, the present invention provides methods for isolating self-renewing centroacinar and terminal ductal progenitors from adult human pancreas. In a specific embodiment, the method comprises the steps of (a) providing a population of pancreatic cells; and (b) selecting for high expression of CD133, EpCAM, and CD44 on the pancreatic cells to isolate self-renewing centroacinar and terminal ductal progenitors.

### STATEMENT OF GOVERNMENTAL INTEREST

This invention was made with U.S. government support under grant no. DK056211. The U.S. government has certain rights in the invention.

### FIELD OF THE INVENTION

The present invention relates to the field of pancreatic progenitor cells.

### BACKGROUND OF THE INVENTION

Although the mammalian pancreas is characterized by steady turnover of differentiated cell types and displays a significant capacity for regeneration following injury, the presence or absence of a dedicated adult pancreatic progenitor population remains controversial. A variety of cell types have been proposed as possible pancreatic progenitors, including preexisting acinar cells (1-3), preexisting 13-cells (4, 5), cells associated with ductal epithelium (6, 7), and mesenchymal-like nestin-expressing cells (8). Despite work suggesting that differentiated pancreatic cell types can act as facultative progenitors, additional studies continue to suggest the presence of more-dedicated progenitor cells in adult pancreas (7).

In addition to the cell types listed above, cells known as centroacinar cells have also been considered as possible multilineage pancreatic progenitors. This poorly characterized cell type lies at the junction between acinar cells and the adjacent terminal ductal epithelium, and it is uncertain whether centroacinar and terminal duct cells represent two different cell types or are functionally equivalent. These cells send out projections that contact both endocrine and exocrine cells (9), and have been shown to rapidly proliferate following partial pancreatectomy (10), streptozotocin administration (11), or administration of caerulein (12). Recent work has also identified centroacinar and terminal duct cells as unique domains of activated Notch signaling in adult human, mouse, and zebrafish pancreas (13-16).

Despite considerable interest in these cell populations, the successful isolation of centroacinar/terminal duct cells has not previously been reported, and their progenitor capacities have never formally been assessed.

### SUMMARY OF THE INVENTION

The present invention is based, at least in part, on the first ever isolation of progenitor cells from adult human pancreas based on a unique combination of surface markers. Beginning with exocrine-enriched “leftovers” of cadaveric human pancreas preparations, in which the corresponding endocrine islet-rich, exocrine-poor fractions have been utilized for islet cell transplantation or research, the present inventors have applied a panel of unique features, as assessed by flow cytometry, to isolate adult human pancreatic progenitor cells. The present invention further demonstrates that these cells can serve as an expandable source of insulin-expressing cells. In addition, the present invention described the development of a novel human pancreatosphere assay to determine the ability of candidate pancreatic progenitor cells to undergo proliferative self-renewal. The present assay can be used to screen for small molecule and genetic modifiers of progenitor expansion and beta cell differentiation.

As described in more detail herein, the present invention comprises methods for the isolation of self-renewing centroacinar and terminal ductal progenitors from adult human pancreas. The successful identification of this novel cell population emanated from iterative cycles of fluorescence-activated cell sorting (FACS) using refined surface markers, followed by bioinformatic analyses to inform additional refinement of sorting strategies. This has resulted in the development of a novel seven-marker panel for isolating human pancreatic progenitor cells including WGA, CD133, CD49f, EpCAM, CD44, CD24 and E-cadherin. Using this panel, the present inventors have identified and isolated a novel cell population comprising less than about 1% of all pancreatic cells, capable of pancreatosphere formation in exocrine-rich fractions of adult human pancreas. In certain embodiments, these adult human pancreatic progenitor cells carry the phenotype Aldefluor(pos)/EpCAM(hi)/CD44(hi)/WGA(low)/CD133(hi)/CD49 (low). The cells are characterized by high level ALDH1 activity, high levels of EpCAM and CD44 expression and low levels of binding by WGA, a lectin with selective affinity for pancreatic acinar cells. In other embodiments, CD133 serves as a functionally equivalent surrogate for EpCAM, and that low CD49 expression can substitute for WGA as a means to exclude exocrine acinar cells. These cells are also characterized by low side (SSC) and forward scatter (FSC). The present inventors have further observed that human pancreatosphere-forming cells have the ability to initiate spontaneous endocrine differentiation, as evidenced by labeling for either insulin C-peptide or glucagon.

Accordingly, in one aspect, the present invention provides methods for isolating self-renewing centroacinar and terminal ductal progenitors from adult human pancreas. In a specific embodiment, the method comprises the steps of (a) providing a population of pancreatic cells; and (b) selecting for high expression of CD133, EpCAM, and CD44 on the pancreatic cells to isolate self-renewing centroacinar and terminal ductal progenitors. In certain embodiments, the selecting step is performed using fluorescence-activate cell sorting (FACS). In a specific embodiment, the selecting step further comprises selecting for low expression of WGA and CD49f. In another embodiment, the cells are gated by forward and side scatter to eliminate debris and aggregates prior to step (b). In certain embodiments, the cells are selected for Aldefluor labeling prior to step (b). In specific embodiments, human antibodies to the specified markers are used to select the target cells.

In another aspect, the present invention also provides a population of cells produced by the methods described herein. In a specific embodiment, the present invention provides a population of cells comprising at least about 80% self-renewing centroacinar and terminal ductal progenitor cells. In a more specific embodiment, the progenitor cells have the phenotype CD133^{high}, EpCAM^{high}, CD44^{high}, CD24^{high} and E-cadherin^{high}. In another embodiment, the progenitor cells have the phenotype WGA^{low} and CD49f^{low}.

In yet another embodiment, a method for isolating self-renewing centroacinar and terminal ductal progenitors from adult human pancreas comprises the steps of (a) providing a population of pancreatic cells; (b) gating cells by forward and side scatter using FACS; and (c) selecting for Aldefluor positive cells to isolate self-renewing centroacinar and terminal ductal progenitors. In one embodiment, the method further comprises selecting for WGA^{low} cells. In another embodiment, the method further comprises selecting for CD44^{high} cells. In a further embodiment, the method further comprises selecting for EpCAM^{high} cells. In yet another embodiment, the method further comprises selecting for CD133^{high} cells. The method may further comprise selecting for CD49f^{low} cells. In one embodiment, the method further comprises selecting for CD44^{high} cells. In another embodiment, the method further comprises selecting for CD24 cells. In a further embodiment, the method further comprises selecting for E-cadherin cells. In yet another embodiment, the method further comprises selecting for WGA^{low} and CD44^{high} cells. Alternatively, the method may further comprise selecting for WGA^{low}, CD44^{high}, and EpCAM^{high} cells.

In another aspect, the present invention provides methods for treating diabetes in a subject in need thereof. In certain embodiments, a method of treating diabetes in a subject comprises transplanting into the subject a population of self-renewing centroacinar and terminal ductal progenitor cells made by the methods described herein. In other embodiments, a method for treating diabetes in a subject comprises the steps of (a) culturing a population of cells made by the methods described herein under conditions that differentiate the progenitors into beta cells; and (b) transplanting the beta cells into the subject.

### BRIEF DESCRIPTION OF THE FIGURES

FIG. 1. ALDH1 expression in embryonic and adult mouse pancreas. (A-C) Immunofluorescent labeling for ALDH1 protein (green) in combination with E-cadherin (red) to mark epithelial structures in E12.5 (A), E14.5 (B and B′), and adult mouse pancreas (C). Image in (B′) represents higher magnification view of area indicated by box in (B). Note restriction of ALDH1 expression to tips of epithelial branches (indicated by asterisks in B′) and not more-central branch trunks (indicated by star). In adult pancreas (C), ALDH 1 expression is restricted to a subset of E-cadherin-positive centroacinar cells. (D and E) Immunohistochemical detection of ALDH1 protein (brown) in subsets of centroacinar (arrows) and terminal duct cells (arrowhead). Scale bars: 50 μM.

FIG. 2. FACS isolation of ALDH1-expressing centroacinar/terminal ductal epithelial cells using the Aldefluor reagent. FACS sorting was performed on single cells isolated from peripheral acinar-ductal units depleted of endocrine and large duct elements. (A and B) Gating of Aldefluor-positive cells based on DEAB-sensitive ALDH1 enzymatic activity. y axis indicates side scatter; x axis indicates intensity of Aldefluor signal (A) with and (B) without DEAB. (B and C) Detection of ALDH1 enzymatic activity (C) with and (D) without DEAB, in conjunction with surface detection of E-cadherin protein. y axis represents intensity of labeling with APC-conjugated anti-E-cadherin antibody; x axis indicates intensity of Aldefluor signal. FACS-sorted populations indicated by P2, P3, P4, and P5 in D correspond to Aldefluor-positive, Ecadherin-negative (A+E−), Aldefluor-positive, E-cadherin-positive (A+E+), Aldefluor-negative, E-cadherin-positive (A−E+), and Aldefluor-negative, E-cadherin-negative (A−E−), respectively. (E and F) Imaging of collagenase-digested mouse pancreas using Aldefluor reagent confirms centroacinar/terminal ductal localization of Aldefluor-positive cells, similar to that observed for ALDH1 immunofluorescence (FIGS. 1 and 2). Note centroacinar/terminal ductal position and small size of Aldefluor-positive cells relative to larger acinar cells, which are easily identifiable by granular cytoplasm corresponding to apical zymogen granules. (Scale bars: 50 μM.) (G) Quantitative RT-PCR analysis of gene expression in A+E+ cells (red), A+E− cells (white), A+E− cells (blue), and A−E− cells (black). Compared with A−E+ aldefluor-negative epithelial cells, A+E+ aldefluor-positive centroacinar/terminal ductal epithelial cells are enriched for transcripts encoding Aldh1a1, Aldh1a7, Sca1, Sdf1, c-Met, Nestin, Ptf1a, and Sox9. Scale bars: 50 μM.

FIG. 3. Formation, differentiation, and function of pancreatospheres derived from Aldefluor-positive centroacinar/terminal ductal cells. (A and B) A+E+ centroacinar/terminal ductal epithelial cells, but not A−E+ epithelial cells, efficiently form pancreatospheres in suspension culture. (C-G) Expression of E-cadherin (C), insulin C-peptide (D), amylase (E), Sox9 (F), and ALDH1 (G) in day 7 pancreatospheres formed from A+E+ centroacinar/terminal ductal epithelial cells. (H) Cell proliferation in day 7 pancreatospheres as assessed by overnight incorporation of EdU added on day 6 of culture period. (I) ELISA-based assay of stored and secreted insulin C-peptide following overnight incubation of either pancreatospheres or Ins-1 cells in varying concentrations of glucose. Note that pancreatospheres display glucose sensitivity similar to that observed in Ins-1 cells (i.e., ˜2-fold increase in secreted C-peptide in response to 0 vs. 11 mM glucose). Scale bars: 100 nM.

FIG. 4. Aldefluor-positive adult pancreatic cells enter both endocrine and exocrine lineages in cultured embryonic pancreas. (A) Schematic of experiment. To trace the lineage of adult cells, Aldefluor (+) and Aldefluor (−) cells were isolated from adult CAG: mCherry transgenic mouse pancreas, microinjected into microdissected dorsal pancreatic buds isolated from E12.5 non-transgenic mouse embryos, and assayed for an ability to productively contribute to the developing endocrine and exocrine lineages. (B-J) Coexpression of mCherry and insulin C-peptide (B-E) and mCherry and glucagon (F-I) confirms capacity of adult Aldefluor (+) cells to contribute to embryonic β- and α-cell lineages, whereas labeling of individual mCherry-positive cells with FITC-conjugated PNA (J-M) confirms ability to contribute to the embryonic acinar lineage. (N) Frequencies with which residual mCherry-positive adult Aldeflouor (+) and Aldefluor (−) cells label for insulin C-peptide, glucagon, Ecadherin, and PNA 7 days after microinjection into microdissected E12.5 dorsal pancreatic buds. All cell counts were determined using E-cadherin labeling to outline the boundary of individual cells. Note that the capacity for endocrine differentiation is predominantly limited to the Aldefluor (+) population, whereas both Aldefluor (+) and Aldefluor (−) cells can productively contribute to the developing exocrine lineages. Scale bars: 50 nM.

FIG. 5. Expansion of ALDH1-expressing centroacinar and terminal ductal epithelial cells in setting of chronic inflammation and regenerative epithelial metaplasia. Following antigen retrieval, ALDH1 protein was detected using immunohistochemistry on pancreatic tissue from normal adult pancreas (A and B) and pancreas harvested from mice with chronic pancreatitis induced by three weekly injections of caerulein (C-H). (A and B) Low-frequency labeling for ALDH1 in terminal ductal (TD) epithelial cells from normal adult pancreas. (C and D) Expansion of ALDH1-expressing terminal ductal epithelium following sequential caerulein administration. (E and F) Similar expansion of ALDH1-expressing centroacinar cells (CAC) following sequential caerulein administration. (G and H) Expression of ALDH1 in caerulein-induced metaplastic type 2 (TC2; H), but not type 1 (TC1; G) tubular complexes.

FIG. 6. Additional presence of ALDH1-positive, E-cadherin-negative mesenchymal cells in adult mouse pancreas.

FIG. 7 Detection of transcripts for insulin (A and B) and Ngn3 (B) by qRT-PCR. (A) Quantification of insulin transcripts in freshly sorted Aldefluor-positive, E-cadherin-positive (A+E+; red), Aldefluor-negative, E-cadherin-positive (A−E+; gray), Aldefluor-positive, E-cadherin-negative (A+E−; blue), Aldefluor-negative, E-cadherin-negative (A−E−; black), and total pancreas (green). Note marked depletion of insulin expression in all four sorted cell fractions, confirming marked depletion of islets in preparations of peripheral acinar-ductal units used for cell sorting. (B) Sequential activation of Ngn3 and insulin expression in pancreatospheres formed from A+E+ cells.

FIG. 8. Adult ALDH1-expressing cells are localized at the junction of terminal ductal epithelium and exocrine acini. Following collagenase digestion and isolation of terminal acinar-ductal units, whole-mount immunofluorescent labeling was performed for ALDH1 protein (red) in combination with E-cadherin (white) and FITC-conjugated DBA to mark terminal ductal epithelium (A, A′, A″, B, B′, B″) or FITC-conjugated PNA to mark the apical membrane of acinar cells (C, C′, C″, D, D′, D″). Note that E-cadherin-positive ALDH1-expressing cells are located in centroacinar and terminal ductal positions. Scale bars: 50 μM.

FIG. 9. Images of living ALDH1-expressing cells within peripheral acinar-ductal units isolated from collagenase-digested mouse pancreas. ALDH1 enzymatic activity is revealed by labeling with the Aldefluor reagent (green). Note terminal ductal/centroacinar position (A and B), as well as positive membrane labeling for E-cadherin (C and D). Images in C′ and D′ correspond to images in C and D, with ALDH1 labeling removed.

FIG. 10. FACS-sorted Aldefluor (+), E-cadherin (+) and Aldefluor (−), E-cadherin (+) epithelial cells display differential expression of ALDH1, Sox9, and amylase protein as assessed by immunofluorescent labeling of cytospin preparations.

FIG. 11. Rates of pancreatosphere formation 7 days following plating of FACS-sorted Aldefluor-positive, E-cadherin-positive (A+E+), Aldefluor-negative, E-cadherin-positive (A−E+), Aldefluor-positive, E-cadherin-negative (A+E−), and Aldefluor-negative, E-cadherin-negative (A−E−) cells.

FIG. 12. Fluorescence-activated cell sorting (FACS) strategy for the isolation of pancreatosphere-forming cells from adult human pancreas. (A): cells (31238 total events) are first gated by forward and side scatter to eliminate debris and aggregates. (B): The resulting population (29520 events) is then gated according to labeling with Aldefluor and WGA. Excluded exocrine cells (A+/WGA+) are boxed in red. (C): the resulting A+/WGA− population (1763 events) is then gated based on labeling for CD44 and EpCAM. This combination allows delineation of two distinct clusters, CD44+/EpCAM+ cells (named here A+E+, light blue circle) and CD44−/EpCAM− cells (named here A+/M+, light tan circle). (D): the strong correlation between CD44 and EpCAM staining allows histogram to be presented based only on CD44 signal alone. Note that the final numbers of A+/E+ cells in panel C and D are identical (A+E+, 436/31238=1.4%; A+M+, 1030/31238=3.3%). (E): pancreatospheres formed from human A+E+ cells. (F): Low-efficiency initiation of endocrine differentiation within human pancreatospheres. Approximately 1% of cells in cultured pancreatospheres initiate expression of either insulin C-peptide or glucagon. (G): Rare C-peptide-expressing cells also express Chromogranin A and nuclear Pdx1. H indicates Hoechst nuclear counterstain.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. DEFINITIONS

The term “pancreatic cell” refers to a pancreatic islet, acinar, centroacinar, duct cell, or any other cell that is a component of the tissue in a developing or mature pancreas. Pancreatic islet cells include alpha, beta, delta, PP, and epsilon cells. Pancreatic cells or pancreatic progenitor cells can include a combination of cells found in the pancreas or of cells that develop or can develop into pancreatic tissue.

As used herein, the terms “specific binding,” “selective binding” and the like, are used interchangeably and refer to a binding reaction which is determinative of the presence of a marker, such as CD44 or EpCAM, in a heterogeneous population of proteins, proteoglycans, and other biologics. Thus, under designated conditions, the antibodies or fragments thereof of the present invention bind to a particular marker or marker fragment or variant thereof without binding in a significant amount to other proteins, proteoglycans, or other biologics present in the subject or sample.

The concept of selective binding to an antibody can involve the use of an antibody that is selected for its specificity for a particular protein, proteoglycan, or variant, fragment, or protein core thereof. A variety of immunoassay formats may be used to select antibodies that selectively bind with a particular protein, proteoglycan, or variant, fragment, or protein core thereof. For example, solid-phase ELISA immunoassays are routinely used to select antibodies selectively immunoreactive with a protein, proteoglycan, or variant, fragment, or protein core thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem. 107:220 (1980).

By a substantially pure population of cells is meant that the cells having a selected phenotype (e.g., self-renewing pancreatic progenitor cells) constitute at least about 85% of the cell population. In more specific embodiments, the cells having the selected phenotype comprise at least about 86%, at least about 87%, at least about 88% at least about 89% or more of the cell population. In another specific embodiment, a substantially pure population of cells refers to cells having a selected phenotype constituting at least about 90% of the cell population. In more specific embodiments, the cells having the selected phenotype comprise at least about 91%, at least about 92%, at least about 93%, at least about 94%, at least about 95%, at least about 96%, at least about 97%, at least about 98%, at least about 99% or 100% of the cell population.

When values are expressed as approximations, by use of the antecedent about, the particular value is disclosed as well. The endpoints of each of the ranges are significant both in relation to the other endpoint, and independently of the other endpoint. Furthermore, where specific values are explicitly disclosed herein, that value, as well as about that value, are disclosed even if not explicitly stated. For example, if the value 10 is explicitly disclosed, then about 10 is also disclosed. When a value is explicitly disclosed, less than or equal to the value, greater than or equal to the value and possible ranges between values are also disclosed. For example, if the value 10 is disclosed then less than or equal to 10, as well as greater than or equal to 10 is also disclosed. It is also understood that, throughout the application, data are provided in a number of different formats, and these data represent endpoints and starting points, and ranges for any combination of the data points. For example, if a particular data point 10 and a particular data point 15 are disclosed, it is understood that greater than, greater than or equal to, less than, less than or equal to, and equal to 10 and 15 are considered disclosed as well as any the range between 10 and 15.

Optional or optionally, as used throughout, means that the subsequently described event or circumstance can, but may not occur, and that the description includes instances where said event or circumstance occurs and instances where it does not.

As used herein, a detectable moiety is any means for detecting an interaction between a marker and its binding moiety, thereby identifying the presence of the marker. The detectable moiety may be detected using various means of detection. The detection of the detectable moiety can be direct provided that the detectable moiety is itself detectable, such as, for example, in the case of fluorophores. Alternatively, the detection of the detectable moiety can be indirect. In the latter case, a second or third moiety reacts or binds with the detectable moiety. For example, an antibody that binds the marker can serve as an indirect detectable moiety to which a second antibody having a direct detectable moiety specifically binds.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the term also includes mammals diagnosed with diabetes.

As used herein, the term “effective,” means adequate to accomplish a desired, expected, or intended result. More particularly, a “therapeutically effective amount” as provided herein refers to an amount of a population of self-renewing centroacinar and terminal ductal progenitor cells, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of a population of self-renewing centroacinar and terminal ductal progenitor cells, necessary to provide the desired therapeutic effect, e.g., an amount that is effective to prevent, alleviate, or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.).

Specific types/examples of antibodies include polyclonal, monoclonal, humanized, chimeric, human, or otherwise-human-suitable antibodies. “Antibodies” also includes any fragment or derivative of any of the herein described antibodies.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### II. CELL POPULATIONS, COMPOSITIONS, AND KITS

Provided herein are populations of pancreatic progenitor cells. Such progenitor cells can optionally give rise to both exocrine and endocrine cells. The described cell populations therefore include populations of centroacinar and terminal ductal progenitor cells.

An example population comprises at least about 80% centroacinar and terminal ductal progenitor cells, including, for example, at least about 80%, at least about 85%, at least about 90%, at least about 95%, at least about 96%, at least about 97%, at least about 98%, at least about 99% or 100% centroacinar and terminal ductal progenitor cells.

The cell populations can be relatively devoid (e.g., containing less than about 20, 15, 10, 9, 8, 7, 6, 5, 4, 3, 2, or 1%) of other cells types such as exocrine cells. Optionally, example cell populations are substantially pure populations of centroacinar and terminal ductal progenitor cells.

In specific embodiments, the centroacinar and terminal ductal progenitor cells are positive for or express high amounts relative to a control of a CD44 marker, for an EpCAM marker, or for both a CD44 marker and an EpCAM marker. By positive for a particular marker, for example, CD44, is meant that CD44-specific antibodies or other specific binding moieties selectively bind to the marker, such that anti-CD44 antibodies or other binding moieties can be used in cell isolation and enriching procedures.

In an example population, the centroacinar and terminal ductal progenitor cells are positive for a CD44 marker and the CD44 positive cells are negative for a WGA marker. In an example population, the progenitor cells are positive for CD44, EpCAM, CD133, CD24 and E-cadherin, and negative or low amounts are present relative to a control for the WGA and CD49f markers. In an example population, the progenitor cells are positive for Aldefluor and negative/low for WGA, and further positive/high for both CD44 and EpCAM. Further provided is a population of centroacinar and terminal ductal progenitor cells, wherein at least about 80% of the centroacinar and terminal ductal progenitor cells are positive for CD44, EpCAM, CD133, CD24 and E-cadherin and negative/low for WGA and CD49f.

In certain embodiments, the centroacinar and terminal ductal progenitor cells of a described population are negative for or express low amounts relative to a control of WGA. In other embodiments, the centroacinar and terminal ductal progenitor cells of a population are negative for or express low amounts relative to a control of CD49f. In further embodiments, the centroacinar and terminal ductal progenitor cells of a population can be negative for both of these markers.

The centroacinar and terminal ductal progenitor cells of a population can be positive or express high amounts relative to a suitable control of CD44, EPCAM, CD133, CD24, E-cadherin or combinations of the foregoing. Thus, the centroacinar and terminal ductal progenitor cells of a population can be CD44 positive (or CD44^{high}). The CD44^{high} cells in a population can negative/low for WGA, CD49f or both. Similarly, the CD44^{high} cells can be positive/high for EPCAM, CD133, CD24, E-cadherin or any combination.

A selected population of the centroacinar and terminal ductal progenitor cells can be optionally cultured under conditions that cause differentiation thereof. The described populations of centroacinar and terminal ductal progenitor cells can be optionally expanded in culture to increase the total number of cells.

Furthermore, the populations of centroacinar and terminal ductal progenitor cells can be immortalized. Immortalized cells include cell lines that divide repeatedly in culture. Immortalized cells are optionally developed by genetic modification of a parent cell. Moreover, the populations of centroacinar and terminal ductal progenitor cells can be genetically modified to express a protein of interest. For example, the cell can be modified to express an exogenous targeting moiety, an exogenous marker (for example, for imaging purposes), or the like. The centroacinar and terminal ductal progenitor cells of the populations can be modified to overexpress an endogenous targeting moiety, marker or the like.

In certain embodiments, the cell populations are cryopreserved. Various methods for cryopreservation of viable cells are known and can be used. See, e.g., Mazur, 1977, Cyrobiology 14:251-272; Livesey and Linner, 1987, Nature 327:255; Linner, et al., 1986, J. Histochem. Cytochem. 34(9):1123-1135; U.S. Pat. No. 4,199,022 to Senkan et al.; U.S. Pat. No. 3,753,357 to Schwartz; U.S. Pat. No. 4,559,298 to Fahy, which are incorporated by reference at least for the methods and compositions described therein).

Also provided herein are kits that include reagents that can be used in practicing the methods disclosed herein and kits comprising the cell populations taught herein. The kits can include any reagent or combination of reagents that would be understood to be required or beneficial in the practice of the disclosed methods. For example, the kits can include cell populations, as well as the buffers or compositions required to use them. Other examples of kits, include reagents for cell sorting and or detection, optionally with buffers, antibodies or compositions required to use them. The kits can also include centroacinar and terminal ductal progenitor cells and instructions to use the same in the methods described herein.

Also provided herein are populations of centroacinar and terminal ductal progenitor cells made or isolated by the methods taught herein.

### III. METHODS FOR IDENTIFYING AND ISOLATING A POPULATION OF CENTROACINAR AND TERMINAL DUCTAL PROGENITOR CELLS

Methods for identifying the markers that characterize self-renewing centroacinar and terminal ductal progenitor cells can be based on any number of methods known in the art. Among the various methods for detecting cells expressing a specific marker, some methods are typically used if the cells are to remain viable following detection, such as for further in vitro study or for transplantation or implantation into a patient, and other methods render the identified cells less amenable to further uses in their living state, for example, in studying pathology specimens or for studies at the termination of cell based or in vivo studies. The methods herein are not so limiting and applications maintaining the viability of living cells as well as those preserving cells are fully embodied herein.

Methods of identifying the markers that characterize the progenitor cells of the present invention can be based on, by way of non-limiting examples, localizing or quantitating marker epitopes on the surface of the cells, or localizing or quantitating marker epitopes within the cytoplasm or subcellular compartments therein. Exemplary methods for the aforementioned localizing or detecting are provided below but are not intended to be limiting in any way.

Detection methods are in one embodiment based upon the detection of the binding of a binding partner to a cell expressing a marker described herein (e.g., CD44, EPCAM, CD133, CD44 and the like). Binding partners can be detectably labeled, or can be unlabeled but further detectable by another binding partner that is detectably labeled and binds thereto. Such uses of binding partners such as antibodies, including labeled primary antibodies and labeled lectins are known in the art. Moreover, combination systems of unlabeled primary antibodies and labeled secondary antibodies are also well known in the art. Such dual systems can also include two antibodies, lectins, avidin-biotin systems, antibodies to labels, and include amplification systems to increase the detection signal. As will be described below, such detection systems are useful not only for identifying the expression of a gene product but also in isolating cells expressing such a gene product utilizing selective binding to a matrix such as a resin or beads. The invention is not so limiting as to the means for detecting the expression of the marker(s) described herein and is inclusive of all such means.

Antibody-based detection methods are among those typically but not always used to identify expression of a protein or an epitope thereof by cells, regardless of whether cells require viability during or after detection. The antibody can be a monoclonal or polyclonal antibody. Ready guidance from the literature can be followed to prepare such antibodies that specifically bind to a marker(s) on the cell surface, and can be used on living cells to detect markers on the cell surface, or in sectioned cells or tissue specimens to detect markers on the surface.

In one embodiment, the term “antibody” includes complete antibodies (e.g., bivalent IgG, pentavalent IgM) or fragments of antibodies in other embodiments, which contain an antigen binding site. Such fragment include in one embodiment Fab, F(ab′)₂, Fv and single chain Fv (scFv) fragments. In one embodiment, such fragments may or may not include antibody constant domains. In another embodiment, F(ab)'s lack constant domains which are required for complement fixation. scFvs are composed of an antibody variable light chain (V_{L}) linked to a variable heavy chain (V_{H}) by a flexible linker. scFvs are able to bind antigen and can be rapidly produced in bacteria. The invention contemplates antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies useful in the present invention can be monoclonal antibodies (Mab) in one embodiment, or polyclonal antibodies in another embodiment. Antibodies which are useful for the methods described herein can be from any source, and in addition may be chimeric. In one embodiment, sources of antibodies can be from a chicken, mouse, rat, sheep, goat, horse, or a human in other embodiments. Secondary antibodies are typically antibodies that bind to another antibody, and are typically prepared in a species different from the originating species of the primary antibody, such that, for example, the secondary antibody may be a rat anti-mouse antibody, or a goat anti-rat antibody, or vice versa, e.g., mouse anti-rat antibody. In some cases a secondary antibody may be directed against a moiety conjugated to the primary antibody, such as a fluorescent moiety. In other embodiments, other binding partners such as avidin and biotin may be employed. In certain embodiments, a detectable primary antibody is used in the detection. In other embodiments, and in particular where amplification of the detectable signal that indicates the presence of the marker is needed, secondary antibodies or even further amplification techniques can be used to increase the detectability of the extent of binding of the primary antibody can be employed. Such amplification systems are well known in the art.

The detection agent described herein can be a lectin or combination of lectins selected or designed to specifically bind to a marker, e.g., the CD133 glycan structure. These lectins can be in solution, detectably labeled or detected or retrieved by a secondary detection antibody or preferably, be attached to a solid substrate such as a magnetic bead or other surfaces that can be used to retrieve cells.

Detection of antibody binding to a cell typically requires a detectable label, either directly bound to the marker-binding antibody (primary antibody) itself, or the detectable label can be present on a secondary antibody that binds to the primary antibody. Various detectable labels are embodied herein, and the selections are not intended to be limiting. Labels such as fluorescent moieties, radioactive elements and compounds, and proteins or other entities with enzymatic activity have been used in the art and are well known, and are applicable to different methods of detection. In one embodiment, among useful fluorescent labels is phycoerythrin. In another embodiment, radioactive labels include ¹²⁵I.

As noted above, the term “detectable label” or “detectably labeled” refers in one embodiment to a composition or moiety that is detectable by spectroscopic, photochemical, biochemical, immunochemical, electromagnetic, radiochemical, or chemical means such as fluorescence, chemifluoresence, or chemiluminescence, or any other appropriate means. In another embodiment, detectable labels are fluorescent dye molecules, or fluorophores, such fluorescein, phycoerythrin, CY3, CY5, allophycocyanine, Texas Red, peridenin chlorophyll, cyanine, FAM, JOE, TAMRA, TET, and VIC, by way of non-limiting examples.

For example, Miltenyi Biotec (Auburn, Calif.) sells antibody-based reagents for identification and isolation of CD133 expressing cells; antibodies include clone AC133 (mouse IgG1), 293C3 (mouse IgG2b), and AC141 (mouse IgG1). These antibodies recognize two different epitopes CD133/1 (clone AC133) and CD133/2 (clone 293C3 and clone AC141), respectively, on the CD133 molecule. Antibody-based reagents for the identification and isolation of the other markers described herein are also commercially available.

Thus, in one embodiment, a labeled primary antibody that binds to a marker, or a combination of an unlabeled primary antibody that binds to a marker and a labeled secondary antibody that binds to the unlabeled primary antibody, can be used to identify marker expressing cells. In another embodiment, a phycoerythrin-conjugated antibody to a marker is used. Using a fluorescent label such as phycoerythrin (PE), marker-expressing cells can be identified using fluorescence microscopy. In other embodiments, a biotinylated primary antibody and a detectable reagent that binds to biotin, such as a fluorescent- or enzyme-conjugated streptavidin or other avidin derivative, can be used for fluorescence localization, immunohistochemical localization or detection by light microscopy. As will be seen below, an advantage of using phycoerythrin is that it is both detectable (fluorescent), and an antibody can be raised thereto, the anti-phycoerythrin antibody useful as an affinity reagent to isolate cells to which phycoerythrin is bound, via for example using the aforementioned phycoerythrin-conjugated anti-marker antibody. The anti-phycoerythrin antibody can be of the same species or of a different species as the primary anti-marker antibody.

In yet another embodiment, localization of marker-expressing cells in a cellular or tissue sample can be performed using immunohistochemical techniques whereby, for example, whole cells or thin sections of tissue are stained with reagents that identify marker epitopes, such as antibodies as described above either directly labeled or by using a labeled secondary antibody that produces a visible product, for example, through an enzymatic reaction, at the sites of the marker. Such immunohistochemical localization methods are well known in the art and can be readily applied to the markers described herein.

Sources of pancreatic cells for the methods described herein include pancreatic islet preparation, i.e., cells isolated from the islets of human or other species pancreata, or cells prepared from human pancreatic tissue. Tissues from adults as well as those from fetal sources are embraced herein. Pancreatic islet cell preparations, which comprise islets and exocrine tissue, can be obtained from any of a number of academic and/or clinical islet purification services. For patients undergoing pancreatectomy for the purpose, for example, of treatment of pancreatitis, the patient's own resected pancreas tissue can provide the source of cells from which pancreatic endocrine progenitor cells can be isolated by the methods embodied herein then administered to the same patient, or to another patient for the treatment of, for example, diabetes mellitus. And likewise, a pancreatectomy patient can be administered autologous pancreatic endocrine progenitor cells from a single unrelated individual or a pool of individuals.

In one embodiment, pancreatic endocrine progenitor cells herein are cells from the adult human pancreas that express EpCAM^{hi}. In another embodiment, the pancreatic progenitor cells herein are cells from the adult human pancreas that express WGA^{low}. In another embodiment, the pancreatic progenitor cells herein are cells from the adult human pancreas that express CD44^{hi}, WGA^{low}, CD133^{hi}, and CD49f^{low}.

The aforementioned exemplary methods for identifying cells expressing the markers described herein, and in particular methods that do not impact the viability of the cells, readily lend themselves to methods for isolating from a mixed cellular population cells that express the markers. Thus, in another embodiment, marker-expressing cells are isolated from or enriched within a mixed cellular population, utilizing various methods of detecting the expression of the markers on the cell surface. By way of non-limiting examples, fluorescence activated cell sorting technology can be used. The various reagents mentioned above useful for identifying cells expressing marker(s) in pancreatic tissue are also useful as reagents for separating such cells from a mixed cellular population, such as by binding to a solid matrix or using magnetic bead technology. Anti-marker antibodies are but one example of the use of a marker binding partner for isolating or separating marker-expressing cells.

Thus, in one embodiment, fluorescence activated cell sorting (FACS) techniques can be used to isolate cells expressing the marker(s), using either a primary anti-marker antibody conjugated to a fluorescent moiety, or an unlabeled or nonfluorescently-labeled primary anti-marker antibody and a secondary antibody conjugated to a fluorescent moiety or a fluorescent reagent that binds to the primary antibody or by using a lectin that recognizes a marker glycan (e.g., CD133). Other binding pairs such as biotin and avidin can be used to achieve the same desired cell labeling. FACS methodology is well known in the art.

In another embodiment, cells expressing CD133 can be directly isolated from a mixed population using a matrix or surface to which an antibody to a marker is conjugated, such that marker-expressing cells bind to the matrix or surface, non-adherent cells can be washed away, and the marker-expressing cells eluted from the matrix or surface. In one embodiment, a matrix such as agarose or Sepharose in the form or beads can be conjugated with antibodies to a marker. Marker-expressing cells in a mixed population are exposed to the matrix, by admixing therewith or passage through a column thereof, to which marker-expressing cells adhere, then the matrix can be washed and the cells eluted therefrom using a high salt or low pH elution buffer, or other methods that interfere with antibody-epitope interaction or methods that act to cleave the connection between the bead and desired cell type. Such methods and reagents therefor are well known in the art. In another embodiment, magnetic beads to which anti-marker antibodies are conjugated are used to bind marker-expressing cells, after which the beads are separated based on their magnetic properties, washed and the marker-expressing cells eluted therefrom. Such magnetic beads are available from Miltenyi Biotec, and methods of use described in the manufacturer's instructions. In yet another embodiment, agarose or Sepharose beads to which lectins are attached are used to bind marker-expressing cells (e.g., CD133-expressing cells). In such embodiments, CD 133 expressing cells in a mixed population are exposed to the matrix, by admixing therewith or passage through a column thereof, to which CD133 expressing cells adhere, then the matrix can be washed and the cells eluted therefrom using an unconjugated glycan to that interferes with the CD133-lectin interaction or methods that act to cleave the connection between the bead and desired cell type. Such methods and reagents therefor are well known in the art

In other embodiments, matrix or magnetic bead separation can be achieved using a secondary antibody conjugated to the matrix or beads, the secondary antibody directed against a primary antibody that binds to a marker. For example, in one embodiment, after use of a primary antibody that binds to a marker that is labeled with phycoerythrin, magnetic beads or a matrix conjugated with an antibody that binds to phycoerythrin can used to bind marker-expressing cells, after which the beads can be washed and the marker-expressing cells released. For example, Miltenyi Biotec sells magnetic beads conjugated to an anti-phycoerythrin antibody (Anti-PE microbeads). Alternately, a secondary antibody against the primary antibody molecule can be used. There methods are merely illustrative of affinity procedures and variations thereof are well known in the art and are fully embraced herein.

In embodiments of the methods for isolation of or enrichment for self-renewing centroacinar and terminal ductal progenitor cells from a cellular population, the cellular population can be obtained from a pancreatic islet preparation, or from human pancreatic tissue. As noted above, pancreatic islet cell preparations can be obtained from any of a number of academic and/or clinical islet purification services. Adult as well as fetal tissues are embraced herein.

In any of the embodiments described herein, the isolated or enriched self-renewing centroacinar and terminal ductal progenitor cells expressing cells can be cultured or expanded in vitro prior to any of the various uses described herein, among others, in order to, by way of non-limiting example, expand or increase the population of cells.

In yet another embodiment of the invention, methods are provided for treating a patient having diabetes mellitus using the centroacinar and terminal ductal progenitor cells isolated from a cellular population in accordance with, and by way of non-limiting examples, the embodiments described above, then administering the progenitor cells to the patient. In one embodiment the cells are cultured or expanded in vitro prior to use.

For example, the self-renewing centroacinar and terminal ductal progenitor cells isolated or enriched in accordance with the embodiments herein can be directly injected into the hepatic duct or the associated vasculature of a patient. In another embodiment the cells can be cultured and expanded in vitro prior to injection. Similarly, cells can be delivered into the pancreas by direct implantation or by injection into the vasculature. Cells engraft into the liver or pancreatic parenchyma, taking on the functions normally associated with pancreatic cells, respectively. Moreover, before implantation or transplantation the cell obtained as described herein can be genetically manipulated to reduce or remove cell-surface molecules responsible for transplantation rejection in order to generate universal donor cells. For example, the mouse Class I histocompatibility (MHC) genes can be disabled by targeted deletion or disruption of the beta-microglobulin gene (see, e.g., Zijlstra, Nature 342:435-438, 1989). This allows indefinite survival of murine pancreatic islet allografts (see, e.g., Markmann, Transplantation 54:1085-1089, 1992). Deletion of the Class II MHC genes (see, e.g., Cosgrove, Cell 66:1051-1066, 1991) further improves the outcome of transplantation. The molecules TAP1 and Ii direct the intercellular trafficking of MHC class I and class II molecules, respectively (see, e.g., Toume, Proc. Natl. Acad. Sci. USA 93:1464-1469, 1996); removal of these two transporter molecules, or other MHC intracellular trafficking systems may also provide a means to reduce or eliminate transplantation rejection. Such techniques can be applied to human cells and the corresponding HLA antigens. In another embodiment, the cellular population is obtained from a pancreas HLA matched to the subject.

### IV. METHODS OF TREATMENT

Provided herein are methods of treating diabetes in a subject. The methods can include the step of transplanting into the subject a population of centroacinar and terminal ductal progenitor cells made by the methods taught herein or using a population of centroacinar and terminal ductal progenitor cells taught herein. The methods can also include culturing a selected population of centroacinar and terminal ductal progenitor cells under conditions that cause differentiation thereof. The resulting differentiated cells, or a subset thereof, can then be transplanted into the subject in need of treatment (e.g., diabetes).

The number of progenitor cells or differentiated cells transplanted can range from about 10²-10⁸ at each transplantation (e.g., injection site), depending on the size and species of the recipient. Single transplantation (e.g., injection) doses can span ranges of about 10³-10⁵, about 10⁴-10⁷, and about 10⁵-10⁸ cells, or any amount in total for a transplant recipient patient.

Delivery of the cells to the subject can include either a single step or a multiple step injection. The cellular transplants are optionally injected as dissociated cells but can also be provided by local placement of non-dissociated cells. In either case, the cellular transplants optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated cellular transplant can be a streaming injection made across the entry path, the exit path, or both the entry and exit paths of the injection device (e.g., a cannula, a needle, or a tube). Automation can be used to provide a uniform entry and exit speed and an injection speed and volume. Optionally a multifocal delivery strategy can be used. Such a multifocal delivery strategy is designed to achieve widespread and dense donor cell engraftment throughout the recipient. Injection sites can be chosen to permit contiguous infiltration of migrating donor cells into particular areas.

In yet another embodiment of the invention, methods are provided for treating a patient having diabetes mellitus using the centroacinar and terminal ductal progenitor cells isolated from a cellular population in accordance with, and by way of non-limiting examples, the embodiments described above, then administering the progenitor cells to the patient. In one embodiment the cells are cultured or expanded in vitro prior to use.

For example, the self-renewing centroacinar and terminal ductal progenitor cells isolated or enriched in accordance with the embodiments herein can be directly injected into the hepatic duct or the associated vasculature of a patient. In another embodiment the cells can be cultured and expanded in vitro prior to injection. Similarly, cells can be delivered into the pancreas by direct implantation or by injection into the vasculature. Cells engraft into the liver or pancreatic parenchyma, taking on the functions normally associated with pancreatic cells, respectively. Moreover, before implantation or transplantation the cell obtained as described herein can be genetically manipulated to reduce or remove cell-surface molecules responsible for transplantation rejection in order to generate universal donor cells. For example, the mouse Class I histocompatibility (MHC) genes can be disabled by targeted deletion or disruption of the beta-microglobulin gene (see, e.g., Zijlstra, Nature 342:435-438, 1989). This allows indefinite survival of murine pancreatic islet allografts (see, e.g., Markmann, Transplantation 54:1085-1089, 1992). Deletion of the Class II MHC genes (see, e.g., Cosgrove, Cell 66:1051-1066, 1991) further improves the outcome of transplantation. The molecules TAP1 and Ii direct the intercellular trafficking of MHC class I and class II molecules, respectively (see, e.g., Toume, Proc. Natl. Acad. Sci. USA 93:1464-1469, 1996); removal of these two transporter molecules, or other MHC intracellular trafficking systems may also provide a means to reduce or eliminate transplantation rejection. Such techniques can be applied to human cells and the corresponding HLA antigens. In another embodiment, the cellular population is obtained from a pancreas HLA matched to the subject.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Dissociation of Adult Mouse Pancreas.

All animal studies were approved by the Animal Care and Use Committee at Johns Hopkins University. Whole adult mouse pancreas was harvested and digested in 1.4 mg/mL collagenase-P (Boehringer Mannheim) at 37° C. for 30 min. Peripheral acinar-ductal units, depleted of large ducts and endocrine islets, were prepared as described previously (22). Following multiple washes with HBSS supplemented with 5% FBS, collagenase-digested pancreatic tissue was filtered through 600 μm and 100 μm polypropylene mesh (Spectrum Laboratories), then spun through a 30% FBS cushion. Peripheral acinar-ductal units were either subjected to whole-mount immunofluorescent labeling or further dissociated for FACS analysis. For FACS, pelleted acinar-ductal units were resuspended in diluted trypsin (0.05%) (Invitrogen) and incubated at 37° C. for 15 min. Dispersed cells were then directly resuspended in Aldefluor buffer.

Immunofluorescent and Immunohistochemical Labeling.

Dissected embryonic pancreas from E10.5-E18.5 embryos or adult mouse pancreas was fixed in 4% paraformaldehyde overnight at 4° C., cryoprotected in 30% sucrose-PBS for 4-6 h at 4° C., OCT embedded and cut into 3- to 4-μm sections. Sections were permeabilized for 15-30 min in 0.2% Triton X-100 in PBS, and blocking of nonspecific reactivity was performed for 1 h in 10% FBS/0.2% TritonX-100 in PBS at RT. Primary antibodies were incubated at the appropriate dilutions in 5% FBS/0.2% TritonX-100 in PBS overnight: rabbit anti-glucagon 1:400 (Novus Biologicals), rabbit anti-ALDH1 1:200 (Abcam), rabbit anti-ALDH1/2 1:200 (Santa Cruz), guinea pig anti-insulin 1:400 (Biomeda), rat anti-E-cadherin 1:400 (Zymed), rabbit anti-Sox9 1:1,000 (Chemicon), goat anti-insulin C-peptide 1:500 (Millipore), and rabbit anti-amylase 1:400 (Sigma). The next morning, slides were washed three times in 0.2% TritonX-100 in PBS, and sections were incubated with the appropriate Cy2- and/or Cy3- and/or Cy5-conjugated secondary IgG antibodies at 1:200 dilution for 1 h at RT in the dark. After three more washes in PBS, nuclei were labeled with DAPI (1:1,000) and slides were mounted in Vectashield mounting medium. Images were acquired using a Zeiss Axiovert imaging microscope. A similar protocol was used for whole-mount immunofluorescent labeling of collagenase digested pancreas. For immunofluorescent labeling of FACS-sorted single cells, 7,000-8,000 sorted cells were pelleted at 1,200 rpm for 3 min onto coated slides using a Shandon Cytospin 4 (Thermo Electron) and dried at room temperature for 5 min before labeling. Immunohistochemical analysis of ALDH1 expression in normal and caerulein-treated adult mouse pancreas was performed as described (15).

Aldefluor Assay and Sorting of Aldefluor-Positive and -Negative Cells by FACS.

The Aldefluor Kit (StemCell Technologies) was used to isolate population with high vs. low ALDH enzymatic activity (hereafter referred to as Aldefluor positive and Aldefluor negative). Dispersed cells resuspended in Aldefluor assay buffer containing ALDH substrate (BAAA, 0.6 ng/μL per 1·107 cells in 1 mL) were incubated for 50 min at 37° C. As a negative control to confirm the specificity of Aldefluor labeling, an aliquot of 5·106 cells from each sample was treated with 1.6 mM diethylaminobenzaldehyde (DEAB), a specific ALDH inhibitor. The sorting gate of the Aldefluor-positive cells was established using DEAB-treated cells as a guide, so that the Aldefluor-positive population was defined by DEAB-sensitive Aldefluor activity. To exclude the possibility that the Aldefluor-positive population was contaminated with endocrine cells, pancreatic tissue was also harvested from Tg(Ins1-DsRed*T4) 32Hara/J mice (5) (obtained from the Jackson Laboratory) and subjected to FACS. Flow cytometry was performed using a FACS Aria (Becton Dickinson) flow cytometer. Labeling with additional antibodies was performed subsequent to Aldefluor staining and without permeabilization. Primary antibody incubations were done in the dark on ice in10% FBS/PBS for 45 min. Following washes, incubation with the secondary antibodies was performed on ice in 5% FBS/PBS for 45 min. The following antibodies were used for flow cytometry: rat anti-E-cadherin (Zymed), rat anti-CD133 (eBioscience), rat anti-Sca-1 (eBioscience), rat anti-Pecam (BD Biosciences), and rat anti-CD45 (BD Biosciences).

RT-qPCR.

Total RNA was prepared using the RNeasy Micro Kit (QIAGEN). RT-qPCR was performed using a C1000 Thermal Cycler Thermo (BioRad) and the IQ SYBR Green SuperMix (BioRad). The PCR volume was 20 μL containing 1.5 μL of diluted cDNA and 250 nM of each primer. Thermocycling conditions included an initial polymerase activation step for 3 min at 95° C., followed by 40 cycles of 30 s at 95° C. for template denaturalization, 30 s at 58° C. for annealing, and 30 s at 72° C. for extension and fluorescence measure. Afterward, a dissociation protocol with a gradient from 65° C. to 95° C. was used for each primer pair to verify the specificity of the RT-qPCR reaction and the absence of primer dimers. In addition, each PCR included a reverse-transcription negative control to check for potential genomic DNA contamination. Reagent contamination was also detected by a reaction mix without template. All samples were amplified in duplicate and normalized against GAPDH as an internal control. The relative quantification of mRNA was performed with theCFX96Real-Time PCR Detection System.

Pancreatosphere Formation Assay.

Pancreatosphere formation assays on sorted Aldefluor-positive and -negative cells were performed by plating cells in 24-well ultra-low attachment plates (Corning) at a density of 6 cells/μL. Cells were grown for 5-7 days in DMEM/F-12 (GIBCO, Invitrogen), 1×N2 Supplement (StemCell Technologies), 20 ng/mL EGF (Peprotech), 20 ng/mL FGF2 (Invitrogen), 1×B27 (StemCell Technologies), 100 nM β-mercaptoethanol (Sigma), 1× nonessential amino acid (Sigma), 1× penicillin/streptomycin (Cellgro), 10 ng/mL LIF (Sigma), and 3% FBS (GIBCO, Invitrogen). For quantitative assays of pancreatosphere formation, cells were sorted directly into 96-well ultra-low attachment plates (Corning) at a density of 1, 10, or 100 cells per well (0.01 cell/nL; 0.1 cell/nL; 1 cell/μL). Serial passages were performed by dissociating spheres using the NeuroCult Chemical Dissociation Kit (StemCell Technologies), selecting viable cells based on trypan blue exclusion, and replating at a density of 6 cells/μL.

Insulin (C-Peptide) Secretion Assays.

Assays of pancreatosphere insulin secretion were performed after 7 days in culture. For comparison, parallel assays were performed on Ins-1 cells (clone 832/13) Pancreatospheres were washed with PBS to eliminate any remaining FBS and then incubated in D-glucose-free RPMI supplemented with 0.25% BSA at three different glucose concentrations (0 mM, 5 mM, and 11 mM). After 12 h, the media was removed, pancreatospheres were lysed in 1 M glacial acetic acid, and insulin C-peptide levels were determined by ELISA (ALPCO). Insulin C-peptide secretion was expressed as a fraction of total cellular C-peptide content.

Injection of Aldefluor-Positive/mCherry Cells in E12.5 Embryonic Pancreas.

E12.5 dorsal pancreatic buds were isolated and injected with 1,000 Aldefluor-positive or -negative cells freshly harvested from adult pCAG:mCherry transgenic mouse pancreas. pCAG:mCherry mice were kindly provided by Michael Wolfgang, Johns Hopkins University. The injected dorsal bud explants were cultured in vitro for 7 days as previously described (35).

### Results

### Example 1

### ALDH1 Expression in Embryonic and Adult Pancreas

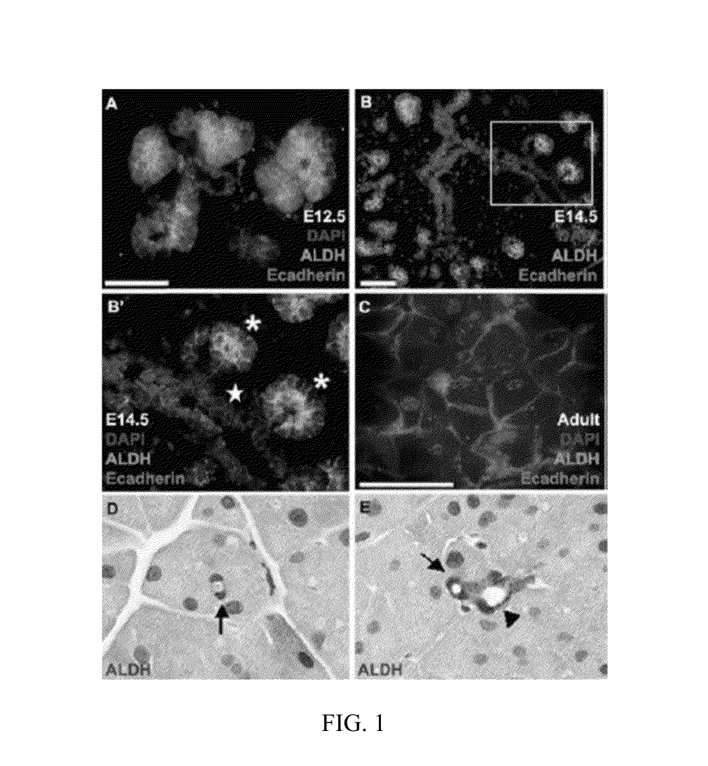

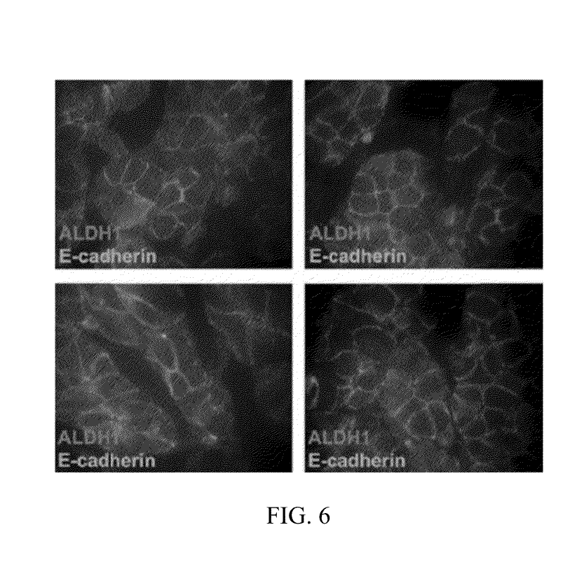

Based on prior studies documenting high levels of ALDH1 enzymatic activity in neural, hematopoietic, and mammary epithelial progenitors (17-19), the temporal and spatial patterns of ALDH1 protein expression were characterized in embryonic and adult mouse pancreas (FIG. 1). Using E-cadherin as a marker of pancreatic epithelial cells, ALDH1 protein was found to be first detectable within the developing pancreatic epithelium on E12.5 (FIG. 1A). At this point, expression is restricted to the tips of the branching tubules, which was recently proposed to represent a multipotential progenitor domain (20). A similar pattern of expression has previously been reported for Aldh1a1 transcripts (21). Expression in the tubular tips (and not in the central trunks) persists through E14.5 (FIGS. 1B and B′), and is subsequently down-regulated in differentiating acinar cells. In adult pancreas, epithelial ALDH1 expression is most frequently observed in centroacinar and terminal ductal epithelial cells (FIG. 1C-E). Mesenchymal (E-cadherin-negative) ALDH1-expressing cells were also detected surrounding endocrine islets and exocrine acini (FIG. 6).

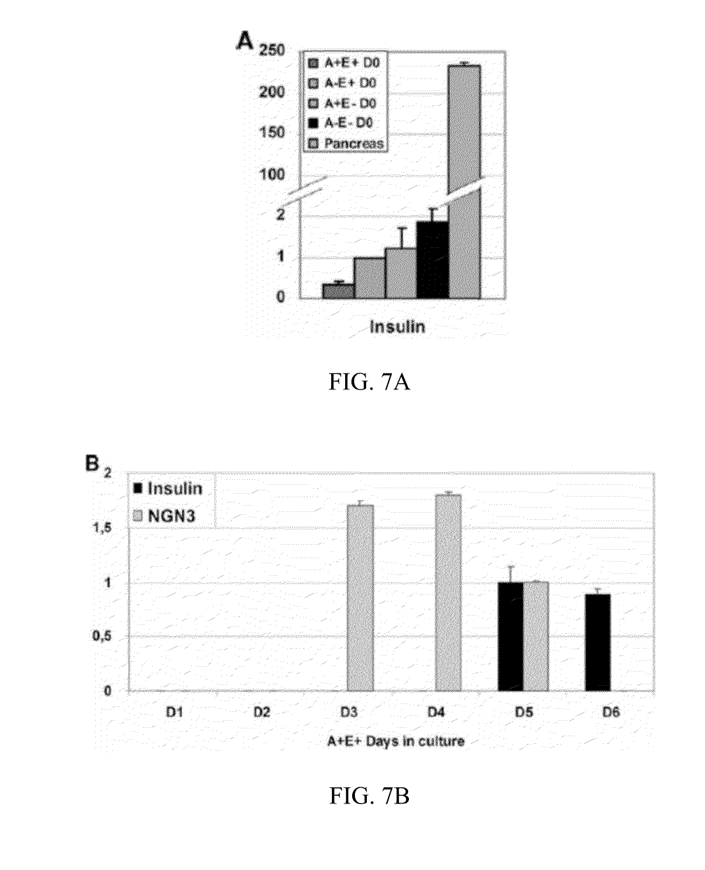

To further characterize ALDH1 expression and ALDH1 enzymatic activity in adult terminal duct/centroacinar cells, preparations of peripheral acinar-ductal units freshly isolated from collagenase-digested mouse exocrine pancreas were used (22). Importantly, these isolated peripheral acinar-ductal units are markedly depleted of large duct and endocrine elements. Compared with total pancreas, peripheral acinar-ductal units exhibited a >400-fold depletion in insulin transcripts, as assessed by RT-PCR (FIG. 7A). When FACS analysis was performed on peripheral acinar-ductal units harvested from transgenic Ins1:DsRed mice expressing red fluorescent protein in β-cells, only 3 of 10,000 cells (0.03%) from this preparation were positive for DsRed.

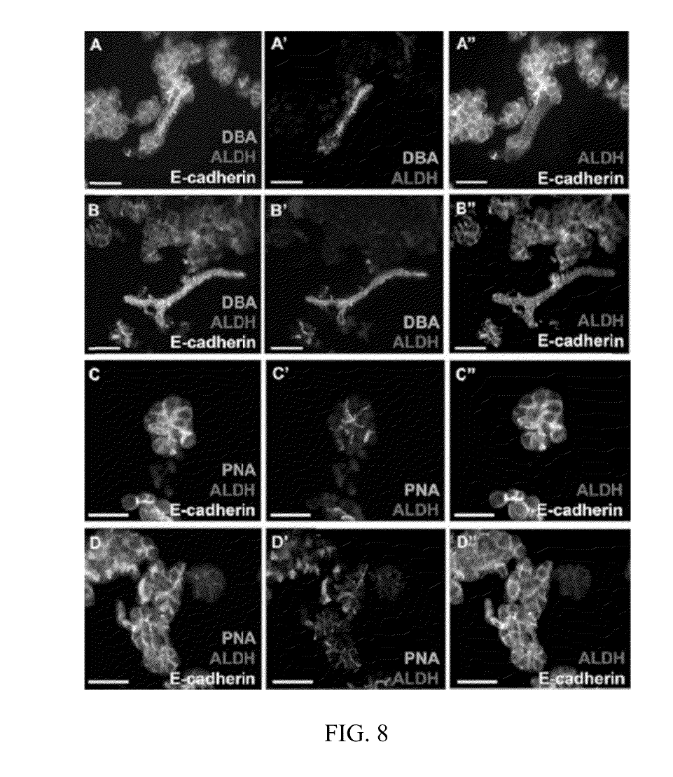

Additional three-dimensional characterization of ALDH1 protein expression was accomplished using whole-mount fluorescent labeling of isolated peripheral acinar-ductal units. ALDH1 protein was localized in combination with E-cadherin as a marker of epithelial cells, and with either FITC-conjugated Dolichos biflorus agglutinin (DBA) or FITC-conjugated peanut agglutinin (PNA), markers of ductal and acinar cells, respectively. Multichannel imaging confirmed a predominantly centroacinar/terminal ductal location of ALDH1-expressing epithelial cells in adult pancreas (FIG. 8). ALDH1-expressing cells were most often interposed between terminal ductal epithelium and more-peripheral acinar cells. In addition, single epithelial ALDH1-expressing cells were also observed immediately adjacent to terminal ductal epithelium (FIGS. 8 A and B). Both DBA-positive and DBA-negative ALDH1-positive cells were identified, whereas ALDH1-positive PNA-positive cells were only rarely identified.

### Example 2

### Isolation of ALDH1-Expressing Centroacinar and Terminal Ductal Cells

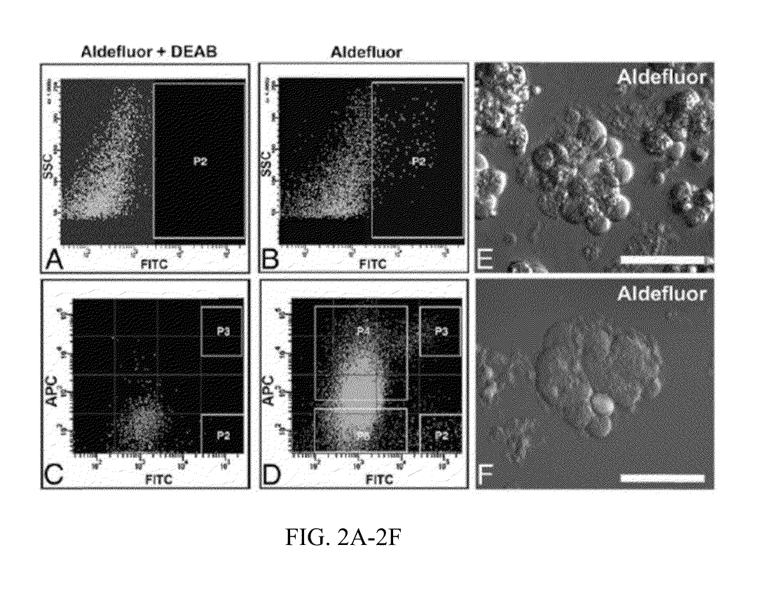

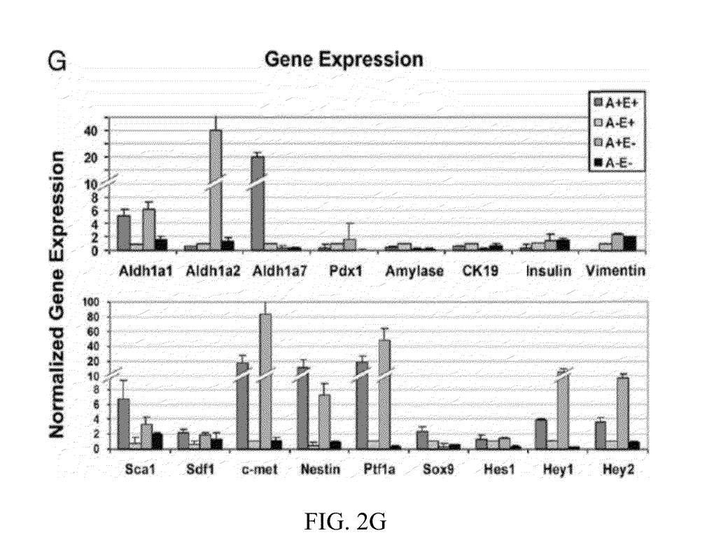

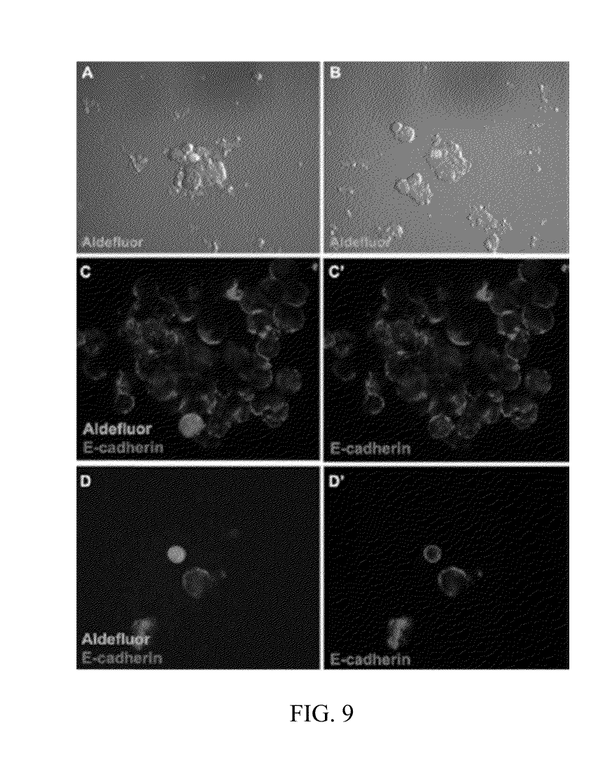

In hopes of isolating ALDH1-expressing cells from adult mouse pancreas, the present inventors took advantage of a fluorogenic substrate known as “Aldefluor” (StemCell Technologies), which has previously been used in the FACS-based isolation of hematopoietic, neural, and mammary epithelial stem cells (17-19). Before attempting FACS-based isolation of ALDH1-expressing pancreatic epithelial cells, this reagent was first used to visualize living ALDH1-expressing cells in peripheral acinar-ductal units (FIG. 2). As shown in FIGS. 2 E and F, these studies confirmed the centroacinar/terminal ductal location of low-abundance Aldefluor-positive cells in adult mouse pancreas. Aldefluor-positive centroacinar/terminal ductal cells were easily distinguished from adjacent acinar cells by virtue of their small size and lack of zymogen granules. Additional examples of live-cell imaging using the Aldefluor reagent are provided in FIG. 9. These findings implied that anti-ALDH1 immunofluorescence and Aldefluor-based cytofluorescence were labeling a similar centroacinar/terminal ductal population, and further suggested that these cells might be successfully isolated by FACS.

FACS-based characterization and sorting of single cells dissociated from peripheral acinar-ductal units was pursued next. As an initial means to establish specific gating of ALDH1-expressing cells, a pharmacologic inhibitor of ALDH1 enzymatic activity (DEAB) was employed. As depicted in FIG. 2 A-D, this strategy allowed for the isolation of a low-abundance cell population characterized by high levels of DEAB-sensitive ALDH1 enzymatic activity, comprising 0.9%±0.2% of all sorted cells in adult mouse pancreas. Using an E-cadherin antibody to simultaneously identify epithelial cells, Aldefluor (+) E-cadherin (+) cells were found to represent 0.5%±0.13% of all sorted cells in adult mouse pancreas (FIG. 2D).

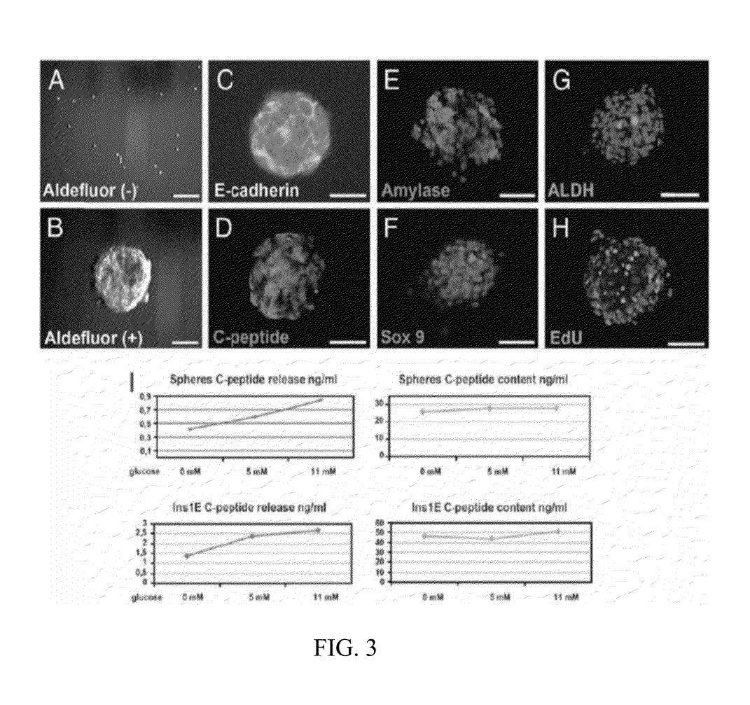

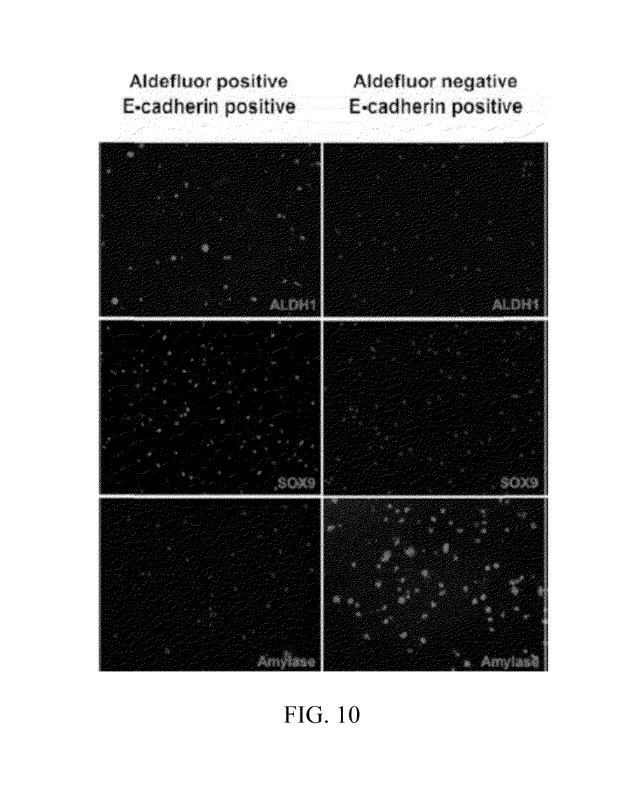

Using quantitative RT-PCR to compare the Aldefluor-positive, E-cadherin-positive (A+E+), Aldefluor-negative, E-cadherin-positive (A−E+), Aldefluor-positive, E-cadherin-negative (A+E−), and Aldefluor-negative, E-cadherin-negative (A−E−) populations isolated from adult mouse pancreas, the A+E+ population was found to be significantly enriched for transcripts encoding Aldh1a1 and Aldh1a7, and depleted of transcripts for two other ALDH1 isoforms, Aldh1a2 and Aldh1a3 (FIG. 3G). Aldh8a1 was not detected in any of the samples. Compared with the A−E+ population, A+E+ cells were modestly depleted of transcripts for Pdx1 (P<0.09), Amylase (P<0.001), and Cytokeratin-19 (P<0.01) (markers expressed in differentiated β-cells, acinar cells, and duct cells, respectively). In contrast, A+E+ cells were characterized by high-level expression of Ptf1a, despite that they were depleted of both Amylase transcripts and amylase protein (FIG. 3G and FIG. 10). In addition, these cells were enriched for transcripts encoding Sca-1, SDF1, c-Met, Nestin, Sox9, Hey1, and Hey2, markers previously associated with progenitor populations in pancreas and other tissues. Using immunofluorescent labeling on cytospin preps of FACS-sorted cells, marked enrichment for ALDH1 and Sox9 protein, and depletion of amylase in A+E+ cells was confirmed (FIG. 10). In addition, FACS analysis was performed to determine the frequency with which Aldefluor (+) cells were also positive for stem cells markers such as CD133 and Sca-1 protein, and observed that over 90% of the Aldefluor (+) cells additionally coexpressed both of these stem cell markers. In contrast, only 0.11% of Aldefluor (+) cells were also positive for the vascular endothelial marker PECAM, whereas 0.08% were positive for the hematopoietic marker CD45. When FACS analysis was performed on peripheral acinar-ductal units harvested from transgenic Ins1-DsRed mice expressing red fluorescent protein in β-cells, all Aldefluor (+) cells were found to be negative for dsRed.

### Example 3

### Pancreatosphere Assay of Endocrine and Exocrine Progenitor Function

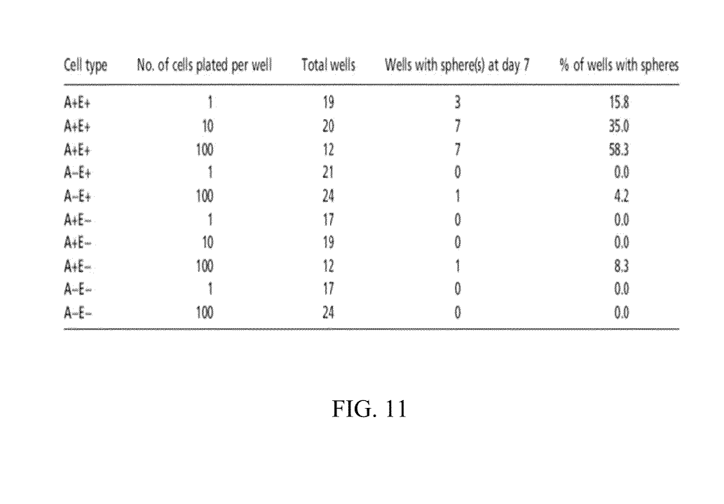

As an initial screen for progenitor-like activity, Aldefluor (+) and Aldefluor (−) cells were assayed for the ability to form pancreatospheres (FIG. 3), similar to the neurosphere assay commonly used to identify neural progenitors (23). In these assays, A+E+ centroacinar/terminal ductal cells were uniquely able to form spheres in suspension culture. A+E+ cells displayed a sphere-forming efficiency >100 times that of their A−E+ counterparts (FIGS. 3 A and B and FIG. 11). With lower efficiencies, single A+E+ cells were even able to form spheres when plated at clonal density (one cell per well) in 96-well plates (FIG. 11). Neither of the E-cadherin-negative populations exhibited significant sphere-forming capacity.

When cultured over a 5- to 7-day period, pancreatospheres derived from A+E+ cells exhibited strong expression of E-cadherin (FIG. 3C), confirming their epithelial identity, and individual cells within the spheres began to accumulate considerable amounts of either amylase or insulin and insulin C-peptide (FIGS. 3 D and E). At 5 days, ≈50% of pancreatospheres displayed expression of amylase, whereas some 30% displayed immunoreactivity to insulin C-peptide. Individual spheres were generally positive for either insulin or amylase, but not both. Small subsets of cells within the spheres maintained ALDH1 expression during the culture period, and also demonstrated nuclear expression of Sox9 protein (FIGS. 3 F and G), suggesting the possible maintenance of a self-renewing progenitor pool. This apparent capacity for self-renewal was further supported by the fact that pancreatospheres generated by Aldefluor (+) centroacinar/terminal ductal cells could be subjected to serial enzymatic dissociation, maintaining their sphere-forming capacity over a minimum of three sequential passages at 7-day intervals. In addition, cells within spheres were highly proliferative, as assessed by overnight incorporation of EdU added at either the beginning or the end of the culture period (FIG. 3H).

Based on the distinct progenitor capacities displayed by A+E+ cells, sorted cell populations were further examined for expression of Ngn3, a marker of endocrine progenitor cells (FIG. 7B). Consistent with previous studies (7), significant expression of Ngn3 in either total adult pancreas or any of the freshly sorted cell populations was not detected. However, once the A+E+ cells were placed in culture, they began to generate detectable expression of Ngn3 immediately preceding the onset of insulin expression, further confirming the endocrine progenitor capacity of ALDH1-expressing centroacinar and terminal ductal epithelial cells.

### Example 4

### Pancreatospheres Derived from Aldefluor (+) Terminal Ductal/Centroacinar Cells Display Glucose-Responsive Insulin Secretion

The detection of cells expressing insulin and insulin C-peptide in cultured pancreatospheres prompted assessment of whether these cells were capable of glucose-responsive insulin secretion, a characteristic of functional β-cells. As a positive control, Ins-1 cells (clone 832/13), an immortalized β-cell line commonly used for studies of insulin secretion in response to physiological concentrations of glucose, were used. Following overnight incubation of either pancreatospheres or Ins-1 cells in 0, 5, and 11 mM glucose, both culture media supernatants and cell lysates were harvested and assayed for secreted and cellular insulin C-peptide using an ELISA-based assay. Pancreatospheres derived from Aldefluor (+) centroacinar/terminal ductal cells secreted C-peptide in a glucose-dependent manner, with glucose sensitivity similar to that displayed by Ins-1 cells (FIG. 3I).

### Example 5

### Aldefluor (+) Adult Terminal Ductal/Centroacinar Cells Can Contribute to Embryonic Endocrine and Exocrine Lineages

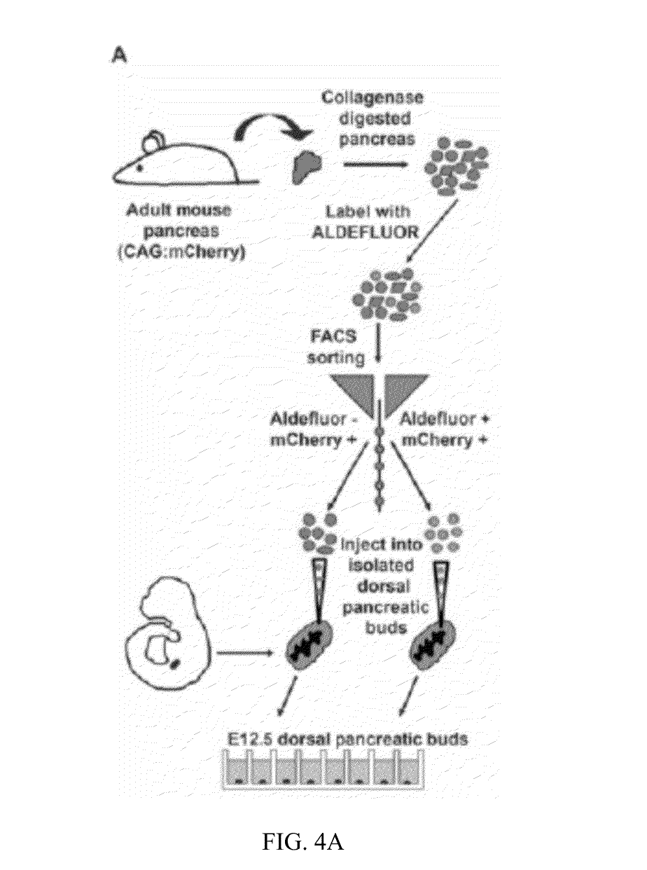

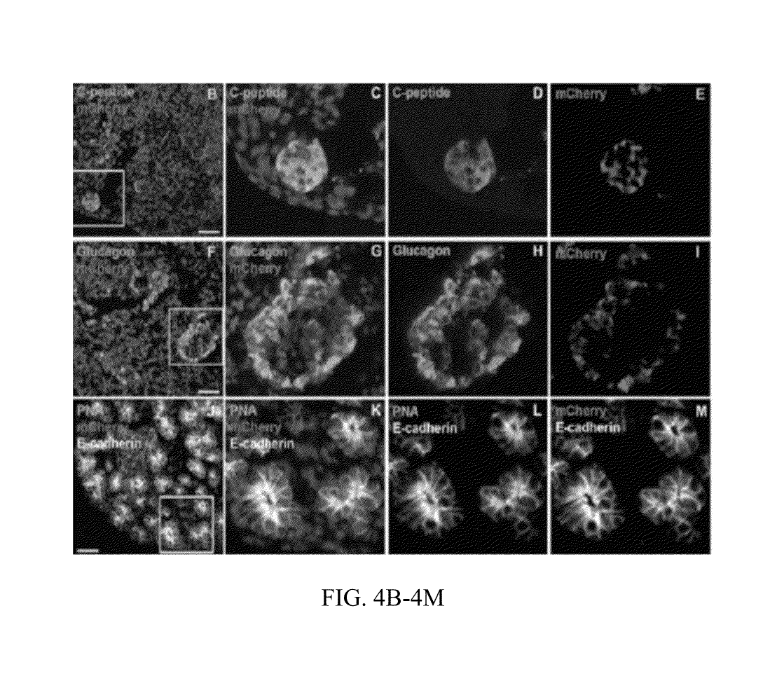

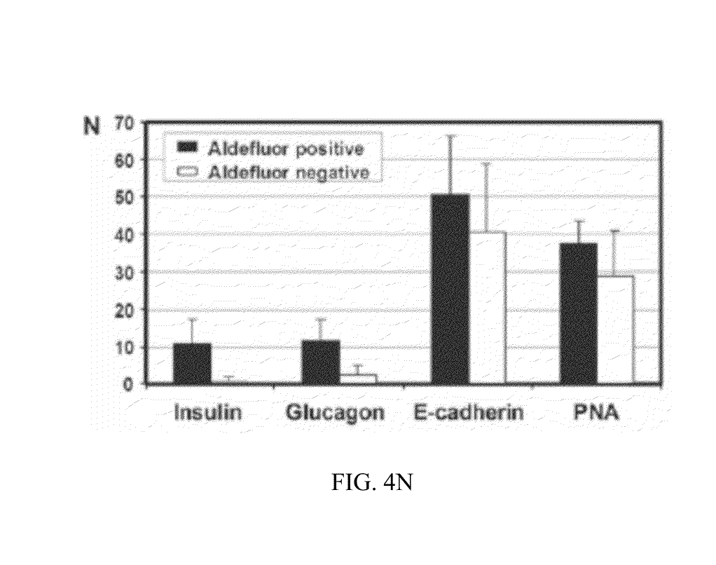

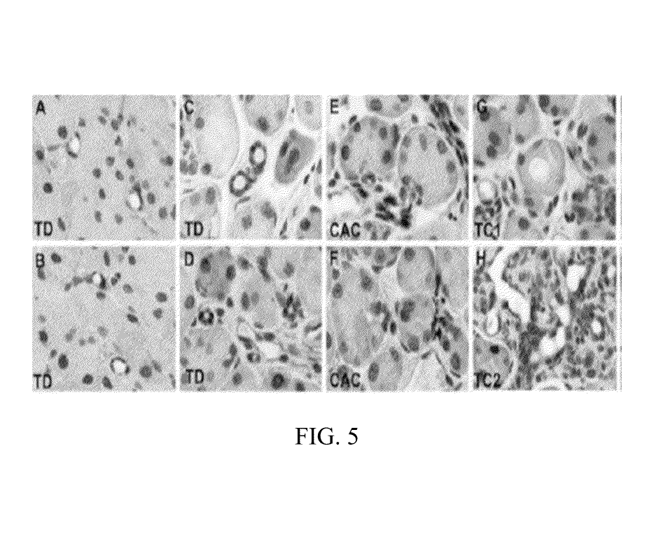

As an even more stringent test for pancreatic progenitor activity, isolated Aldefluor (+) and Aldefluor (−) cells were microinjected into microdissected dorsal pancreatic buds isolated from E12.5 mouse embryos, and assayed for an ability to productively contribute to the developing endocrine and exocrine lineages (FIG. 4). This approach was recently used to document progenitor activity for Ngn3-expressing cells arising following pancreatic duct ligation (7). To trace the lineage of adult-derived donor cells and distinguish them from their embryo-derived counterparts, Aldefluor (+) and Aldefluor (−) cells were isolated from the pancreas of mice carrying a ubiquitously expressed pCAG:mCherry transgene, as schematically depicted in FIG. 4A (pCAG:mCherry mice were kindly provided by Michael Wolfgang, Johns Hopkins University). When compared with Aldefluor (−) cells, Aldefluor (+) cells carried a dramatically enhanced potential to contribute to emerging endocrine lineages within the maturing dorsal buds, as demonstrated by coexpression of mCherry with either C-peptide (FIG. 4 B-E) or glucagon (FIG. 4 F-I). Using superimposed E-cadherin labeling to allow counting of individual mCherry-positive cells, the ability of adult Aldefluor (+) and Aldefluor (−) cells to enter into embryonic lineages was quantitatively evaluated. Seven days following microinjection of Aldefluor (+) cells into E12.5 dorsal buds, expression of glucagon was observed in 11.7% of residual mCherry positive cells, with insulin C-peptide expression observed in an additional 11.6% (FIG. 5N). In contrast, 2.4% of residual mCherry-positive Aldefluor (−) cells expressed glucagon, and only 0.2% expressed insulin C-peptide. Interestingly, the Aldefluor (+) and Aldefluor (−) populations displayed equivalent abilities to enter into non-endocrine epithelial lineages, perhaps reflecting the fact that most of the Aldefluor (−) population was comprised of already differentiated acinar cells. Similar frequencies of E-cadherin, amylase, and PNA positivity were observed in residual mCherry-positive cells derived from either the Aldefluor (+) or Aldefluor (−) populations (FIG. 4 J-N).

### Example 6

### Expansion of ALDH1-Expressing Centroacinar and Terminal Duct Cells Following Chronic Epithelial Injury

To evaluate the in vivo behavior of ALDH1-expressing centroacinar and terminal duct cells, patterns of ALDH1 expression were assessed in the setting of chronic inflammation and regenerative metaplasia induced by sequential administration of low-dose caerulein. As previously reported (15), treatment of adult mice with three injections of caerulein (50 mg/kg) per week for 3 consecutive weeks induced a state of chronic pancreatitis followed by near complete regeneration and repair. This process is characterized by inflammatory infiltrates, stromal expansion, and the formation of regenerative metaplastic tubular complexes, which include type-1 tubular complexes previously shown to be acinar cell-derived, and type-2 tubular complexes (TC2), previously shown to be nonacinar derived and presumably arising from proliferating terminal duct cells (15). In contrast to the relatively low abundance of ALDH1-expressing terminal duct and centroacinar cells observed in normal adult pancreas (FIGS. 5 A and B), ALDH1-expressing terminal duct (FIGS. 5 C and D) and centroacinar (FIGS. 5 E and F) cells were markedly expanded in the setting of caerulein-induced chronic pancreatitis. Of note, type-2 tubular complexes were comprised predominantly of ALDH1-expressing cells (FIG. 5H), whereas type-1 tubular complexes showed no evidence of ALDH1 expression (FIG. 5G).

In normal adult pancreas, it has been shown that progenitor-like cells capable of in vitro endocrine differentiation can be enriched by flow cytometry using a variety of surface markers (24-27). However, a major challenge has been to unequivocally localize these cells in normal pancreas, as the combinatorial application of multiple cell-surface markers, each expressed along a continuous high-low gradient, has often precluded standard immunohistochemical or immunofluorescent approaches. More recently, Ngn3-positive endocrine progenitor cells were visualized as they arose adjacent to terminal ductal epithelium following pancreatic duct ligation (7), suggesting that, under normal conditions, Ngn3-negative multipotent progenitor cells may also reside in this position.

In the present study, a population of cells residing in a centroacinar/terminal ductal position and characterized by unique capacities suggesting progenitor function has been identified. Specifically, these cells express high levels of Ptf1a, Sox9, Sca-1, SDF-1, c-Met, and Nestin. Associated with this unique pattern of gene expression, adult Aldefluor-positive centroacinar and terminal ductal epithelial cells carry a unique capacity to form pancreatospheres, as well as to contribute to both endocrine and exocrine embryonic lineages. Together, these findings suggest that at least a subset of cells residing in a centroacinar/terminal ductal location is capable of progenitor function. Though lineage tracing studies in adult pancreas will be required to determine the actual role played by these cells during normal tissue homeostasis, the finding that this population undergoes dramatic expansion in the setting of chronic epithelial injury suggests that these cells are recruited in the context of pancreatic epithelial regeneration. Together with their location at the junction between peripheral secretory cells and more central ductal epithelium, these characteristics suggest similarity between centroacinar/terminal ductal cells and hepatic oval cells, an injury-responsive progenitor cell type similarly capable of multilineage differentiation (28).

In the present study, high levels of ALDH1 enzymatic activity were exploited purely as a marker of centroacinar/terminal ductal progenitors. These studies therefore do not address whether this enzymatic activity, especially as it relates to synthesis of retinoic acid, plays an important role in the function of these progenitors, or whether they serve as local sources of retinoic acid production in a manner that organizes surrounding cells. Retinoids have previously been shown to exert profound influences on vertebrate pancreas development (29-31), and retinoic acid is a critical component for directed differentiation of human ES cells into insulin-producing β-cells (32). In the hematopoietic system, in vitro inhibition of ALDH1 enzymatic activity has been reported to somewhat paradoxically lead to the expansion of undifferentiated hematopoietic progenitors (33), and studies using gene-targeted mice have suggested that Aldh1a1-specific enzymatic activity is dispensable for hematopoietic and neural progenitor cell activity (34). However, the multiplicity of genes encoding ALDH1 enzymatic activity obviously renders single-gene loss-of-function studies difficult to interpret, and only a subset of ALDH1 family members (Aldh1a1, Aldh1a2, Aldh1a3, and Aldh8a1) appear to carry all-trans retinal dehydrogenase activity (http://www.aldh.org/superfamily.php). In this regard, Aldefluor-positive centroacinar and terminal ductal epithelial cells are characterized by high-level expression of Aldh1a1 and Aldh1a1, and low-level expression of Aldh1a2, Aldh1a3, and Aldh8a1.

Though centroacinar and terminal ductal epithelial cells certainly remain less well-characterized than other pancreatic cell types, the current findings add to an expanding knowledge base regarding these cells. In addition to mounting a proliferative response to various forms of pancreatic injury, centroacinar cells have also been shown to dramatically proliferate following pancreas-specific knockout of Pten, allowing them to act as apparent cells of origin for pancreatic neoplasia (16). Based on immunohistochemical labeling, these cells have also been suggested to undergo in vivo endocrine differentiation following islet injury (11). Relevant to their capacity to act as progenitors, human, mouse, and zebrafish centroacinar cells also appear to be characterized by active Notch signaling (13-16), a feature that they appear to share in common with terminal ductal epithelial cells (15). Surprisingly, Aldefluor-positive centroacinar and terminal ductal epithelial cells did not display up-regulation of Hes1 transcripts, but did exhibit up-regulated expression of Hey1 and Hey2, consistent with an active Notch pathway.

In summary, the present inventors have isolated a unique population of centroacinar and terminal ductal epithelial cells from adult mouse pancreas, and shown that these cells carry significant progenitor capacities. Though additional lineage tracing studies will be required to formally establish these cells as dedicated adult pancreatic progenitors, further characterization and manipulation of this population may prove useful in the treatment of human pancreatic disease.

### Example 7

### FACS Strategy for Isolation of Pancreatosphere-Forming Cells from Adult Human Pancreas

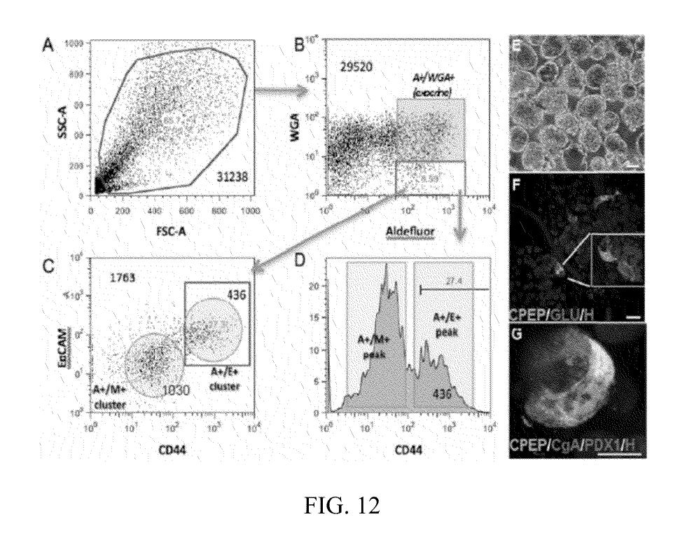

Following on the successful isolation of progenitor cells from adult mouse pancreas, the present inventors committed to applying similar throughput to the identification of self-renewing human adult pancreatic progenitors. Taking advantage of the availability of frequent human pancreas procurements, the present inventors have subjected over 50 such human preps to FACS analysis. In so doing, a novel A+/E+ correlate capable of pancreatosphere formation in exocrine-rich fractions of adult human pancreas has been identified. As shown in FIG. 12, these candidate adult human pancreatic progenitor cells are characterized by high level ALDH1 activity, high levels of EpCAM and CD44 expression, and low levels of binding by WGA, a lectin with selective affinity for pancreatic acinar cells. In additional data not shown, CD133 serves as a functionally equivalent surrogate for EpCam, and that low CD49 expression can substitute for WGA as a means to exclude exocrine acinar cells. As in the case of the original A+/E+ murine population, human pancreatosphere-forming cells have the ability to initiate spontaneous endocrine differentiation.

### REFERENCES

1. Zhou Q, Brown J, Kanarek A, Rajagopal J, Melton D A (2008) vivo reprogramming of adult pancreatic exocrine cells to beta-cells. Nature 455:627-632.
2. Sangiorgi E, Capecchi M R (2009) Bmil lineage tracing identifies a self-renewing pancreatic acinar cell subpopulation capable of maintaining pancreatic organ homeostasis. Proc Natl Acad Sci USA 106:7101-7106.
3. Dor Y, Brown J, Martinez O I, Melton D A (2004) Adult pancreatic beta-cells are formed by self-duplication rather than stem-cell differentiation. Nature 429:41-46.
4. Fendrich V, et al. (2008) Hedgehog signaling is required for effective regeneration of exocrine pancreas. Gastroenterology 135:621-631.
5. Teta M, Rankin M M, Long S Y, Stein G M, Kushner J A (2007) Growth and regeneration of adult beta cells does not involve specialized progenitors. Dev Cell 12:817-826.
6. Inada A, et al. (2008) Carbonic anhydrase II-positive pancreatic cells are progenitors for both endocrine and exocrine pancreas after birth. Proc Natl Acad Sci USA 105: 19915-19919.
7. Xu X, et al. (2008) Beta cells can be generated from endogenous progenitors in injured adult mouse pancreas. Cell 132:197-207.
8. Zulewski H, et al. (2001) Multipotential nestin-positive stem cells isolated from adult pancreatic islets differentiate ex vivo into pancreatic endocrine, exocrine, and hepatic phenotypes. Diabetes 50:521-533.
9. Leeson T S, Leeson R (1986) Close association of centroacinar/ductular and insular cells in the rat pancreas. Histol Histopathol 1:33-42.
10. Hayashi K Y, et al. (2003) Differentiation and proliferation of endocrine cells in the regenerating rat pancreas after 90% pancreatectomy. Arch Histol Cytol 66:163-174.
11. Nagasao J, Yoshioka K, Amasaki H, Mutoh K (2003) Centroacinar and intercalated duct cells as potential precursors of pancreatic endocrine cells in rats treated with streptozotocin. Ann Anat 185:211-216.
12. Gasslander T, Ihse I, Smeds S (1992) The importance of the centroacinar region in cerulein-induced mouse pancreatic growth. Scand J Gastroenterol 27:564-570.
13. Miyamoto Y, et al. (2003) Notch mediates TGF alpha-induced changes in epithelial differentiation during pancreatic tumorigenesis. Cancer Cell 3:565-576.
14. Parsons M J, et al. (2009) Notch-responsive cells initiate the secondary transition in larval zebrafish pancreas. Mech Dev 126:898-912.
15. Strobel O, et al. (2007) In vivo lineage tracing defines the role of acinar-to-ductal transdifferentiation in inflammatory ductal metaplasia. Gastroenterology 133: 1999-2009.
16. Stanger B Z, et al. (2005) Pten constrains centroacinar cell expansion and malignant transformation in the pancreas. Cancer Cell 8:185-195.
17. Storms R W, et al. (1999) Isolation of primitive human hematopoietic progenitors on the basis of aldehyde dehydrogenase activity. Proc Natl Acad Sci USA 96:9118-9123.
18. Ginestier C, et al. (2007) ALDH1 is a marker of normal and malignant human mammary stem cells and a predictor of poor clinical outcome. Cell Stem Cell 1: 555-567.
19. Corti S, et al. (2006) Identification of a primitive brain-derived neural stem cell population based on aldehyde dehydrogenase activity. Stem Cells 24:975-985.
20. Zhou Q, et al. (2007) A multipotent progenitor domain guides pancreatic organogenesis. Dev Cell 13:103-114.
21. Oström M, et al. (2008) Retinoic acid promotes the generation of pancreatic endocrine progenitor cells and their further differentiation into beta-cells. PLoS One 3:e2841.
22. Means A L, et al. (2005) Pancreatic epithelial plasticity mediated by acinar cell transdifferentiation and generation of nestin-positive intermediates. Development 132:3767-3776.
23. Marshall G P, II, et al. (2008) Production of neurospheres from CNS tissue. Methods Mol Biol 438:135-150.
24. Suzuki A, Nakauchi H, Taniguchi H (2004) Prospective isolation of multipotent pancreatic progenitors using flow-cytometric cell sorting. Diabetes 53:2143-2152.
25. Ku H T (2008) Minireview: Pancreatic progenitor cells—recent studies. Endocrinology 149:4312-4316.
26. Sugiyama T, Rodriguez R T, McLean G W, Kim S K (2007) Conserved markers of fetal pancreatic epithelium permit prospective isolation of islet progenitor cells by FACS. Proc Natl Acad Sci USA 104:175-180.
27. Oshima Y, et al. (2007) Isolation of mouse pancreatic ductal progenitor cells expressing CD133 and c-Met by flow cytometric cell sorting. Gastroenterology 132: 720-732.
28. Rountree C B, et al. (2007) A CD133-expressing murine liver oval cell population with bilineage potential. Stem Cells 25:2419-2429.
29. Martin M, et al. (2005) Dorsal pancreas agenesis in retinoic acid-deficient Raldh2 mutant mice. Dev Biol 284:399-411.
30. Molotkov A, Molotkova N, Duester G (2005) Retinoic acid generated by Raldh2 in mesoderm is required for mouse dorsal endodermal pancreas development. Dev Dyn 232:950-957.
31. Stafford D, et al. (2006) Retinoids signal directly to zebrafish endoderm to specify insulin-expressing beta-cells. Development 133:949-956.
32. Kroon E, et al. (2008) Pancreatic endoderm derived from human embryonic stem cells generates glucose-responsive insulin-secreting cells in vivo. Nat Biotechnol 26: 443-452.
33. Chute J P, et al. (2006) Inhibition of aldehyde dehydrogenase and retinoid signaling induces the expansion of human hematopoietic stem cells. Proc Natl Acad Sci USA 103:11707-11712.
34. Levi B P, Yilmaz O H, Duester G, Morrison S J (2009) Aldehyde dehydrogenase 1a1 is dispensable for stem cell function in the mouse hematopoietic and nervous systems. Blood 113:1670-1680.
35. Esni F, Miyamoto Y, Leach S D, Ghosh B (2005) Primary explant cultures of adult and embryonic pancreas. Methods Mol Med 103:259-271.

We claim:

1. A method for isolating self-renewing centroacinar and terminal ductal progenitors from adult human pancreas comprising the steps of:
a. providing a population of pancreatic cells; and
b. selecting for high expression of CD133, EpCAM, and CD44 on the pancreatic cells to isolate self-renewing centroacinar and terminal ductal progenitors.

2. The method of claim 1, wherein the selecting step is performed using fluorescence-activate cell sorting (FACS).

3. The method of claim 1, wherein the selecting step further comprises selecting for low expression of WGA and CD49f.

4. The method of claim 2, wherein the cells are gated by forward and side scatter to eliminate debris and aggregates prior to step (b).

5. The method of claim 2, wherein the cells are selected for Aldefluor labeling prior to step (b).

6. A population of cells produced by the method of claim 1.

7. A population of cells comprising at least about 80% self-renewing centroacinar and terminal ductal progenitor cells.

8. The population of cells of claim 7, wherein the progenitor cells have the phenotype CD133^{high}, EpCAM^{high}, CD44^{high}, CD24^{high} and E-cadherin^{high}.

9. The population of cells of claim 7, wherein the progenitor cells have the phenotype WGA^{low} and CD49f^{low}.

10. A method for isolating self-renewing centroacinar and terminal ductal progenitors from adult human pancreas comprising the steps of:
a. providing a population of pancreatic cells;
b. gating cells by forward and side scatter using FACS; and
c. selecting for Aldefluor positive cells to isolate self-renewing centroacinar and terminal ductal progenitors.

11. The method of claim 10, further comprising selecting for WGA^{low} cells.

12. The method of claim 10, further comprising selecting for CD44^{high} cells.

13. The method of claim 10, further comprising selecting for EpCAM^{high} cells.

14. The method of claim 10, further comprising selecting for CD133^{high} cells.

15. The method of claim 10, further comprising selecting for CD49f^{low} cells.

16. The method of claim 10, further comprising selecting for CD44^{high} cells.

17. The method of claim 10, further comprising selecting for CD24 cells.

18. The method of claim 10, further comprising selecting for E-cadherin cells.

19. The method of claim 10, further comprising selecting for WGA^{low} and CD44^{high} cells.

20. The method of claim 10, further comprising selecting for WGA^{low}, CD44^{high}, and EpCAM^{high} cells.

21. A method of treating diabetes in a subject comprising transplanting into the subject a population of self-renewing centroacinar and terminal ductal progenitor cells made by the methods of any one of claims 1.

22. A method for treating diabetes in a subject comprising the steps of:
a. culturing a population of cells made by the methods of claim 1 under conditions that differentiate the progenitors into beta cells; and
b. transplanting the beta cells into the subject.
